(19) United States
(12) Patent Application Publication
(10) Pub. No.: US 2021/0395747 A1
(43) Pub. Date: Dec. 23, 2021

(54) METHODS AND COMPOSITIONS FOR TREATMENT OF FRONTOTEMPORAL DEMENTIA

(71) Applicant: Saint Louis University, St. Louis, MO (US)

(72) Inventor: Andrew Tuan Duc Nguyen, St. Louis, MO (US)

(73) Assignee: Saint Louis University, St. Louis, MO (US)

(21) Appl. No.: 17/346,369

(22) Filed: Jun. 14, 2021

Related U.S. Application Data
(60) Provisional application No. 63/041,619, filed on Jun. 19, 2020.

(51) Int. Cl. C12N 15/113; A61K 9/00; A61P 25/28

(52) U.S. Cl. CPC C12N 15/1136; A61K 9/0019; C12N 2320/35; C12N 2310/321; C12N 2310/315; A61P 25/28

(57) ABSTRACT

Disclosed are methods and compositions for treating cognitive impairment due to frontotemporal dementia caused by progranulin mutations. More specifically, the invention relates to antisense oligonucleotides (ASOs) and their administration to subjects to increase progranulin protein levels in brain tissue, to treat cognitive impairment due to frontotemporal dementia caused by progranulin mutations.

### CROSS REFERENCE TO RELATED APPLICATION

This application claims priority to provisional application 63/041,619, filed Jun. 9, 2020, hereby incorporated by reference in its entirety.

### GOVERNMENT SUPPORT CLAUSE

The work disclosed herein was supported by the NIH/National Center for Advancing Translational Sciences grant UL1TR002345 (award no. CTRFP2019-11). The U.S. Government has certain rights in this invention.

### FIELD OF THE INVENTION

The invention relates to methods and compositions for treating reduced cognitive ability due to frontotemporal dementia. More specifically, the invention relates to the use of antisense oligonucleotides to inhibit negative control elements of progranulin expression and increase progranulin levels in cells, for the purpose of treating subjects with frontotemporal dementia caused by progranulin mutations.

### BACKGROUND

Frontotemporal dementia, is a devastating disease with a mean survival of 3.8 years from diagnosis. No cure is currently available. It is caused by mutations in one of the two alleles of the progranulin gene which leads to a 50% decrease in progranulin protein levels in the brain, blood, cerebrospinal fluid and throughout the body etc. This decrease in progranulin levels causes frontotemporal dementia. As a disease of haploinsufficiency, strategies aimed at increasing progranulin levels are considered feasible therapeutic approaches. A number of academic labs and pharmaceutical companies are carrying out compound screens and genetic screens to identify methods and compounds that will increase progranulin levels. Some compounds identified so far include histone deacetylase inhibitors such as SAHA, which have been found to be very nonspecific and affect proteins besides progranulin and trehalose, which is unstable, and therefore requires a high dose to increase progranulin levels in mice. Alternatively, a gene therapy approach has been successfully conducted in mice, and provides proof-of-concept studies that increasing progranulin levels is beneficial in a mouse model of frontotemporal dementia (Arrant et al., (2017) Brain: 140; 1447; Arrant et al., (2018) J. Neurosci, February 28,•38(9):2341). Most recently, Alector, is developing a monoclonal antibody that targets a sorting receptor to increase progranulin levels by altering its trafficking/stability. However, off-target effects are a major concern with this strategy because the receptor is known to bind to many other proteins in addition to progranulin.

Because antisense oligonucleotides (ASOs) target the mRNA in a sequence-specific manner, they are generally quite specific and lower doses can be used compared to other therapies, such as antibody-based therapies. Through efforts to modify the ASO chemistry, current ASOs are quite stable in vivo. In humans, ASOs are safe and well tolerated, including when delivered to the brain. Several ASO drugs including Nusinersen, Eteplirsen, Fomivirsen, and Mipomersen have been approved by the US FDA.

The Inventor has developed antisense oligonucleotides (ASOs) that increase levels of the progranulin in cells of the brain. More specifically, these ASOs negate negative control elements (microRNA) of progranulin translation by targeting specific microRNA binding sites on progranulin mRNA. As a result, these ASOs can be used to increase levels of the progranulin in cells of the brain. The Inventor believes that these ASOs will increase progranulin levels in humans and provide a therapy for cognitive decline associated with frontotemporal dementia, which currently has no cure.

### SUMMARY OF THE INVENTION

An antisense oligonucleotide (ASO) for increasing progranulin in cells, which binds to a miR-29b-3p binding site within the progranulin mRNA, and increases progranulin expression when delivered into a brain cell for treatment of frontotemporal dementia.

An antisense oligonucleotide for increasing progranulin in cells, which binds to a miR-659-3p1′ or miR-659-3p2′ binding site within the progranulin mRNA, and increases progranulin expression when delivered into a brain cell for treatment of frontotemporal dementia.

The ASOs of the invention further comprising modifications of 2′-O-methoxyethyl nucleotides and phosphorothioate linkages.

A method of treating for frontotemporal dementia by administering via intrathecal or intercerbreal injection of an ASO which binds to a miR-29b-3p or miR-659-3p2′ binding site within human progranulin mRNA, thereby increasing progranulin protein expression when delivered to a brain cell.

### DESCRIPTION OF THE FIGURES

FIG. 1 is a schematic representation of the human GRN mRNA and miR-659 and miR-29b binding sites in the 3′ untranslated region (UTR). ‘A series’ ASOs target the miR-659 binding site where as ‘M series’ ASOs target the miR-29b binding site. Single strand oligonucleotide like symbols above the mRNA represent approximate binding positions of the A and M series ASOs. The beginning and ending nucleotide positions are indicated below the mRNA.

FIG. 2 Illustrates that antisense oligonucleotides (ASOs) will increase progranulin protein levels in human neurons. The indicated ASOs were delivered alone and in combination to differentiated SY5Y human neurons in culture at a concentration of 100 nM, using Lipofectamine 2000. After two days, levels of the protein progranulin was determined by western blot analysis. Tubulin is shown as a control to indicate even protein loading (bottom). ASO B-2 in particular was shown to increase progranulin.

FIG. 3 Illustrates the analysis of additional ASOs tested on differentiated SY5Y human neurons in culture using the methods described in FIG. 2. ASO A-3 in particular was shown to increase progranulin, as was a number of ASOs in combination.

FIG. 4 Illustrates increased progranulin protein levels in human H4 neuroglioma cells treated with the A series and M series ASOs. A) H4 cells were treated with 5 μM ASOs for 24 h and progranulin protein levels measured by ELISA. B) Validation of ASO ELISA results were done by western blot (10 μM, 24 h).

FIG. 5 Illustrates increase of progranulin protein levels of select ASOs in a dose-dependent manner in H4 neuroglioma cells. Dose curves show an increase progranulin levels after 24 hours of treatment with ASOs M1, M2, M5, M10, M11, M12, and M36. EC₅₀ values are indicated.

FIG. 6. ASOs targeting the miR-29b binding site in the GRN mRNA increase progranulin protein levels in cultured human neurons and in mouse brains. A) ASOs M5 and M11 treatment increases progranulin protein levels after 3 days in induced pluripotent stem cell (iPSC)-derived cortical neurons, in a dose-dependent manner. B) Central administration of ASO M5 increases human progranulin protein levels in the cortex, thalamus and hippocampus, of transgenic mice, engineered to express human progranulin. ASO M5 (500 μg ASO) was administered by bolus intracerebroventricular (ICV) injections and brain tissues were analyzed after two weeks.

### DETAILED DESCRIPTION OF THE INVENTION

In humans with frontotemporal dementia, progranulin mutations occur in one of the two alleles, which leads to an approximately 50% decrease in progranulin protein levels, and which in turn leads to a decline in cognitive ability. Several microRNAs, by way of example, miR-29b, miR-107, and miR-659, have been shown to bind to the progranulin mRNA and decrease progranulin protein levels (Chen et al., (2017) J. Cell. Mol. Med. Vol 21, No 12, 2017 pp. 3347; Jiao (2010) May|Vol. 5|Issue 5|e10551). The present invention relates to antisense oligonucleotides (ASOs), that are complementarity with, and bind to, progranulin mRNA, so as to block the binding sites, and binding thereof, of miR-29b-3p and miR-659-3p. The Inventor has also discovered that it is not necessary for these ASOs occupy the entire binding site, but need only target the vicinity of the binding site. For example, certain ASOs that flank the 5′ end of the binding site or overlap the binding site by 1 or more nucleotides are effective. The ASOs of the invention will block miR-29b-3p and miR-659-3p binding to progranulin mRNA, thereby negating the inhibitory effect of these microRNAs on progranulin mRNA translation, resulting in increased levels of progranulin protein in subjects with frontotemporal dementia. The ASOs were conceived by the Inventor and manufactured according to their specifications by a commercial vendor, Integrated DNA Technologies, with a further chemical modification of 2′-O-methoxyethyl nucleotides and phosphorothioate linkages.

### I. MicroRNAs

miRNAs are small, non-coding 20-24 nt RNAs that promote the silencing of their target genes by binding to specific, partially complementary regions that are often located in the 3′ untranslated regions (UTR) of the target mRNA. This results in RNA interference and/or translational repression of the target gene (for review see Bartel, (2009) Cell; 136:215; Olena, and Patton (2010) J Cell Physiol.; 222:540). miRNAs can be transcribed from their own promoter or may be encoded in the introns of other genes. It is speculated that in the latter case these miRNA might be expressed when the “hosting” mRNA is transcribed. Regardless, microRNAs are transcribed as primary Pri-miRNA (200-400 nt) which are first processed by the exonuclease Drosha resulting in a Pre-miRNA (100-150 nt), which is then exported to the cytoplasm via exportin, a nuclear export factor. Pre-miRNA is then further cleaved by Dicer, a ribonuclease III and its cofactors (PACT and TRBP) to generate a mature miRNA (20-24 nt) (Bartel, (2009) Cell; 136:215; Olena, and Patton, (2010) J Cell Physiol.; 222:540) which contains duplexes of 19 to 25 nucleotides. The double-stranded RNA dissociates and one strand is incorporated into the RNA-induced silencing complex (RISC). The miRNA/RISC complex is then capable of binding to target mRNAs and inhibiting expression through cleavage and degradation of the target mRNA (RNA silencing) and/or by interfering with translation.

### II. Antisense Compounds

RNA interfering systems that include RNA or DNA oligonucleotides that are complementary to a target nucleic acid and are commonly referred to as “antisense” oligonucleotides. Antisense oligonucleotides will be complementary to a chosen target nucleic acid so that the antisense oligonucleotide will specifically hybridize to the target nucleic acid. An antisense therapy requires first identifying a target nucleic acid sequence whose function is to be modulated. In the present invention, the target nucleic acid whose function is to be modulated is the human progranulin mRNA. More specifically, the present invention encompasses antisense oligonucleotides that are at least partially complementary to the binding sites for miR-29b-3p and miR-659-3p of human progranulin mRNA. The function to be modulated is the binding of inhibitory elements miR-29b-3p and miR-659-3p, so as to allow increased translation of progranulin protein.

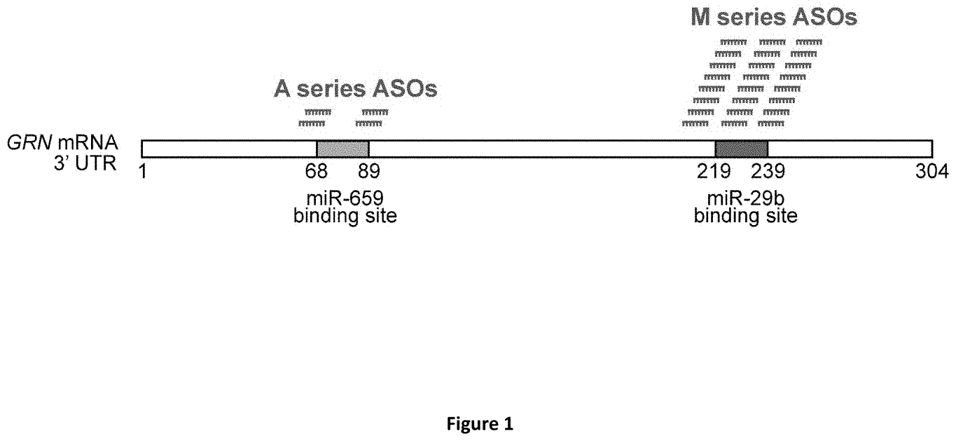

ASOs that target the miR-659 and miR-29b binding sites of the 3′ untranslated region (UTR) of human GRN mRNA are represented in a schematical illustrated in FIG. 1. ‘A series’ ASOs target the miR-659 binding site and ‘M series’ ASOs target the miR-29b binding site. Beginning and ending nucleotide positions of the 3′ UTR are indicated below the RNA. Symbols representing approximate or relative positions of the A and M series ASOs are shown above the GRN mRNA. The sequence of human GRN mRNA may be found in the Sequence Listing as SEQ ID NO:1, herby incorporated by reference, and at NCBI Reference Sequence: NM_002087.4, also incorporated by reference. The 3′ UTR corresponds to nucleotides 1823-2126 of the human GRN mRNA as annotated in NM_002087.4.

The present invention encompasses antisense oligonucleotides including but not limited to the following:

An antisense oligonucleotide targeted to the miR-29b-3p binding site on progranulin mRNA. More specifically an ASO, which binds to progranulin mRNA and preferable 3 or more, or most preferably 4 or more nucleotides of the miR-29b-3p binding region, and which increases translation of progranulin protein. The miR-29b-3p binding site on progranulin mRNA is set forth as: GGACCCUGUGGCCAGGUGCUU (SEQ ID NO:2).

Non-limiting examples of oligonucleotide sequences of ASOs which are complementary to the miR-29b-3p binding site on progranulin mRNA are listed in Table 1.

TABLE 1
″M″ series ASO Sequences

| ASO | Sequence | |
|---|---|---|
| Designation | (5′ to 3′) | SEQ ID NO: |
| M1 | AAACGGGGAGGGGATGGC | SEQ ID NO: 3 |
| M2 | GAAACGGGGAGGGGATGG | SEQ ID NO: 4 |
| M3 | TGAAACGGGGAGGGGATG | SEQ ID NO: 5 |
| M4 | CTGAAACGGGGAGGGGAT | SEQ ID NO: 6 |
| M5 | ACTGAAACGGGGAGGGGA | SEQ ID NO: 7 |
| M6 | CACTGAAACGGGGAGGGG | SEQ ID NO: 8 |
| M10 (B3) | GGTCCACTGAAACGGGGA | SEQ ID NO: 9 |
| M11 (B1) | GGGTCCACTGAAACGGGG | SEQ ID NO: 10 |
| M25 | AGCACCTGGCCACAGGGT | SEQ ID NO: 11 |
| M28 | AAAAGCACCTGGCCACAG | SEQ ID NO: 12 |
| M29 | GAAAAGCACCTGGCCACA | SEQ ID NO: 13 |
| M31 | GGGAAAAGCACCTGGCCA | SEQ ID NO: 14 |
| M36 | GGATAGGGAAAAGCACCT | SEQ ID NO: 15 |
| M38 (B6) | GTGGATAGGGAAAAGCAC | SEQ ID NO: 16 |
| M39 (B2) | TGTGGATAGGGAAAAGCA | SEQ ID NO: 17 |
| M40 (B5) | CTGTGGATAGGGAAAAGC | SEQ ID NO: 18 |

An antisense oligonucleotide targeted to the miR-659-3p binding site on progranulin mRNA. More specifically an ASO, which binds to progranulin mRNA and preferable 3 or more, or most preferably 4 or more nucleotides of the miR-659-3p binding region, and which increases translation of progranulin protein. The two miR-659-3p binding sites on progranulin mRNA are set forth as: AGGCCUCCCUAGGCCU (SEQ ID NO:19) and AGCACCUCCCCCUAACCAAA (SEQ ID NO:20) and referred to herein as miR-659-3p1′ and miR-659-3p2′, respectively.

Non-limiting examples oligonucleotide sequences of ASOs which are complementary to the miR-659-3p binding site on progranulin mRNA are listed in Table 2.

TABLE 2
″A″ series ASO Sequences

| ASO | Sequence (5' to 3') | SEQ ID NO: |
|---|---|---|
| A2 | GTGCTAGGGAGGCCTGAG | SEQ ID NO: 21 |
| A3 | GGTCCAGGGAGAATTTGG | SEQ ID NO: 22 |
| A6 | GTCCAGGGAGAATTTGGT | SEQ ID NO: 23 |
| A7 | TGCTAGGGAGGCCTGAGC | SEQ ID NO: 24 |

Two preferred ASOs sequences are designated ASO M39 (B2) and ASO A-3. More preferred ASO are M1, M2, M4, M10, M11, M36 and M38. A most preferred ASO is M5.

M series ASOs listed in Table 1 target the miR-29b-3p binding site. A series ASOs listed in Table 2 target the miR-659-3p2′.

It is appreciated that antisense oligonucleotides may substitute uracil (U) with thymine (T), or thymine (T) with uracil (U) and maintain complementation to the target nucleic acid.

All oligonucleotides sequences disclosed herein are designated from 5′ to 3′, unless otherwise noted.

To be effective, it is not necessary for an antisense oligonucleotide to hybridization 100 percent with the target nucleic acid. Antisense oligonucleotides are chosen which are sufficiently complementary to the target nucleic acids, and which bind sufficiently well and with sufficient specificity, to give the desired effect on the target mRNAs. It is expected that antisense oligonucleotides which are complementary to the entire sequence of one or more of these target nucleic acids will be effective in increasing translation of the target mRNA, resulting in increased protein levels. It is also expected that antisense oligonucleotides which are complementary to less than the entire sequence of one or more of the target nucleic acids will be effective in increasing translation of the target mRNA, and thus increasing protein levels. Antisense oligonucleotides effective in modulating their target mRNAs are expected to be complementary to at least 8, preferably at least 10, more preferably at least 12; more preferably to at least 14; even more preferably to at least 18; yet more preferably to at least 22 nucleic acids of one or more of the target nucleic acids. It is also preferred that the antisense compound hybridize to nucleic acids that are contiguous.

As used herein, the term “antisense oligonucleotide” is meant to include oligonucleotides, with or without modified backbones, and is intended to include related chemical compounds that specifically bind to the same targeted nucleic acids described herein, and provide the same regulatory effect as the subject antisense oligonucleotides.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′,” which may also be expressed as “5′-A-C-T-3′.” The Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second noncomplementary target.

The term “hybridization”, as used herein means hydrogen bonding, which may be Watson-Crick, Hoogsteen or reversed Hoogsteen hydrogen bonding, between complementary nucleotide bases. For example, adenine and thiamine, and guanine and cytosine, respectively, are complementary nucleobases that pair through the formation of hydrogen bonds. “Complementary”, as that term is used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are complementary to each other at that position. The oligonucleotide and the DNA or RNA are complementary to each other when a sufficient number of corresponding positions in each molecule are occupied by nucleotides that can hydrogen bond with each other. “Specifically hybridize” means that a particular sequence has a sufficient degree of complementarity or precise pairing with a DNA or RNA target sequence that stable and specific binding occurs between the oligonucleotide and the DNA or RNA target. It is understood in the art that the sequence of an antisense compound need not be 100% complementary to that of its target nucleic acid to be specifically hybridizable. Typically, for specific hybridization in vitro, moderate stringency conditions are used such that hybridization occurs between substantially similar nucleic acids, but not between dissimilar nucleic acids. In in vitro systems, stringency conditions are dependent upon time, temperature and salt concentration as can be readily determined by the skilled artisan. (See, e.g., Sambrook et al., Molecular Cloning: A Laboratory Manual, Cold Spring Harbor Laboratory Press, NY (1989)). For in vivo antisense methods, the hybridization conditions consist of intracellular conditions which govern the hybridization of the antisense oligonucleotide with the target sequence. An antisense compound specifically hybridizes to the target sequence when binding of the compound to the target DNA or RNA molecule interferes with the normal translation of the target DNA or RNA such that a functional gene product is not produced, and there is a sufficient degree of complementarity to avoid non-specific binding.

The term “modifications of 2′-O-methoxyethyl nucleotides and phosphorothioate linkages,” as used herein are meant to include but not be limited to a modification where the hydroxyl of the 2′ ribose moiety of a nucleoside is substituted with a methoxyethyl group. In addition to, or in the alternative, are modifications that include phosphorothioate linkages that replace phosphodiester linkages between the nucleosides. For a review of oligonucleotide modifications see Schoch and Miller, (2017) Neuron 94, Elsevier Inc, June 21, p 1056-1070, incorporated by reference herein in its entirety.

The terms “deliver” or “delivered” as used herein are meant to include any and all methods of administrating an ASO in any manner that will allow uptake by a cell, including a brain cell, in vivo or in vitro.

One embodiment of the invention is ASO M39 (B2), an oligonucleotide sequence described above, with or without the modifications of 2′-O-methoxyethyl nucleotides and phosphorothioate linkages.

In another embodiment of the invention, is ASO A3 an oligonucleotide sequence described above, with or without the modifications of 2′-O-methoxyethyl nucleotides and phosphorothioate linkages.

In another embodiment of the invention, is ASO M5 an oligonucleotide sequence described above, with or without the modifications of 2′-O-methoxyethyl nucleotides and phosphorothioate linkages.

In another embodiment of the invention, is ASO M11 an oligonucleotide sequence described above, with or without the modifications of 2′-O-methoxyethyl nucleotides and phosphorothioate linkages.

In another embodiment of the invention, is ASO M36 an oligonucleotide sequence described above, with or without the modifications of 2′-O-methoxyethyl nucleotides and phosphorothioate linkages.

In another embodiment is a composition comprising any one or more ASO disclosed herein alone or in combination, with or without the modifications of 2′-O-methoxyethyl nucleotides and phosphorothioate linkages.

In another embodiment, is an antisense oligonucleotide complementary to the nucleic acid sequence of the progranulin mRNA, and which binds to, and at least partially overlap, the miR-29b-3p binding site within the progranulin mRNA, with or without the modifications of 2′-O-methoxyethyl nucleotides and phosphorothioate linkages, and increases progranulin expression when transfected into a cell.

In another embodiment, is an antisense oligonucleotide complementary to the nucleic acid sequence of the progranulin mRNA, and which binds to, and at least partially overlap, the miR-659-3p binding site within the progranulin mRNA, with or without the modifications of 2′-O-methoxyethyl nucleotides and phosphorothioate linkages, and increases progranulin expression when transfected into a cell.

In another embodiment is 5 a composition comprising any one or more ASO disclosed herein alone or in combination prepared in an injectable solution.

In another embodiment is a method of treating a subject with frontotemporal dementia by administering a composition comprising any one or more ASO disclosed herein alone or in combination via bolus intrathecal injections.

In another embodiment is a method of treating a subject with frontotemporal dementia by administering is a composition comprising any one or more ASO disclosed herein alone or in combination by intracerebroventricular injection.

In another embodiment, is an injectable preparation of any one or more of the ASOs described herein, alone or in combination. By way of example, one or more ASO may be prepared any physiological salt solution such as saline. The solution may or may not include a transfection agent, by way of non-limiting example, Invivofectamine. A pharmaceutical acceptable preparation may also include a preservative, a composition to block non-specific binding of the ASO, by way of example a protein such as gelatin or albumin, or a compound, such as a surfactant, by way of example, Tween 20 preferably at 0.1 to 1 percent. ASO solutions may also be prepared in glycerol, liquid polyethylene glycols, or mixtures thereof and pharmaceutically acceptable oils the solution may also include compounds to prevent degradation or aggregation of the ASO while staying within physiological acceptable parameters for injection. By way example, the pH may vary from 5.0 to 6.0, 6.0 to 7.0, 7.0, to 8.0, or 9.0 to 10.0. One or more salts may also be included. Non-naturally occurring salts may also be included, by way of example, Tris, HCL. ASO formulations may be concentrated to some degree or lyophilized for storage and later hydrated before use.

### III. Subjects

It is envisioned that subjects selected for treatment would include human subjects, particularly human subjects diagnosed with, or at risk of, frontotemporal dementia. The ASOs of the invention may be administered to subjects at risk of frontotemporal dementia, prophylactically. Human subjects at risk for frontotemporal dementia would include, by way of example, those with progranulin mutations, decreased progranulin levels in plasma or cerebrospinal fluid, family histories of progranulin-deficient frontotemporal dementia, and/or symptoms of frontotemporal dementia, by way of example, the behavioral variant or primary progressive aphasia. It is expected that symptoms and brain atrophy associated with frontotemporal dementia will be halted or reduced. The effectiveness of ASO treatment may be assessed through neuropsychological examination directed to language, behavior, memory, executive and visual-spatial functions, the Mini-Mental State Examination, neuroimaging-based biomarkers, and/or fluid biomarkers in the blood and cerebrospinal fluid including neurofilament light chain (see Meeter et al., (2016) Ann Clin Transl Neurol. 2016 Jul. 1; 3(8):623; Scherling et al., (2014) Ann Neurol. 2014 January; 75(1):116)

### IV. Treatment

It is expected that treatment with the ASOs of invention will stop or reduce the rate of further cognitive decline in subjects with frontotemporal dementia. Administration of ASOs may be by any method capable of delivering ASOs to the brain. By way of non-limiting example, antisense oligonucleotides may be administered to subjects including humans by bolus intrathecal injections. Antisense oligonucleotides have been shown to be safe and well tolerated in humans with promising results in clinical trials for spinal muscular atrophy, Duchenne muscular dystrophy, ALS, and Huntington's disease. In human trials, antisense oligonucleotide, such as IONIS-HTT_{Rx}/RG6042, have been successfully delivered to the brain via bolus intrathecal injections for treatment of Huntington's disease. (see Tabrizi et al., (2019) N Engl J Med. 2019 Jun. 13; 380(24):2307) Alternatively, the antisense oligonucleotides may be administered by intracerebroventricular (ICV) injections. This method of administration results in widespread distribution of the antisense oligonucleotides throughout the brain (Scoles et al., (2017) Nature 544, 362-366; Becker, et al., (2017) Nature 544, 367-371), and the antisense oligonucleotides typically remain effective for about 3 months (Kordasiewicz, et al., (2012) Neuron 74, 1031-1044; DeVos, et al., (2017) Sci. Transl. Med. 9, eaag0481). Also, Miller et al., conducted Phase 1-2 trials of the antisense oligonucleotide Tofersen, used to treat SOD1 ALS. There were observed effects at 40 mg/patient and higher. (Miller et al., (2020) Engl J MED vol. 383 no. 2, 109-119).

In one non-limiting embodiment, is a human subject, exhibiting evidence of frontotemporal dementia-related cognitive decline, is treated with an intracerebroventricular injection of, ASO B-2 or ASO A-3. An ascending-dose of 10 mg, 30 mg, 60 mg, 90 mg, or 120 mg is anticipated as reported in the IONIS-HTTRx/RG6042 trial (Tabrizi et al., (2019) N Engl J Med., June 13; 380(24):2307). It is also anticipated that subsequent to this initial short-term trial, subject will proceed to a long-term treatment, by way of example, a dose of 120 mg either monthly or every other month, although long-acting formulations injections are envisaged. The optimum dose and frequency for a particular subject may be determined by the patient's treating physician, based on the patient's cognitive response and/or measurement of progranulin protein levels in the blood or cerebrospinal fluid. It is anticipated that ASO therapy may or may not improve a subject's cognitive function relative to current baseline; however, ASO therapy may result in significant delays in the advancement of the disease. Improvement may manifest as a significant delay or a reduced rate of subsequent cognitive decline.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

Materials and Methods

SH-SY5Y neuroblastoma cells and H4 neuroglioma cells were obtained from ATCC and maintained in culture using known methods of cell culture. iPSCs engineered for rapid differentiation into cortical neurons were from Dr. Michael Ward at NIH. These cells were differentiated for 2 weeks as described (PMID: 29924488) prior to ASO treatment. Transfection, was performed according to the manufacturer's protocol (Lipofectamine 2000, from ThermoFisher). Western blot techniques were carried out according to previously published methods (Nguyen et al., (2018) Proc Natl Acad Sci USA. March 20; 115(12):E2849) and anti-progranulin antibodies were provided by the Bluefield Project to Cure Frontotemporal Dementia (Nguyen et al., (2013) J Biol Chem., March 22; 288(12):8627). ELISA assays were carried out according to the manufacturer's instructions (R&D Systems, catalog number DPGRN0). Humanized GRN mice have the human progranulin gene with regulatory elements such as the 3′ UTR inserted at the HPRT locus (PMID: 33636385).

### Example 1

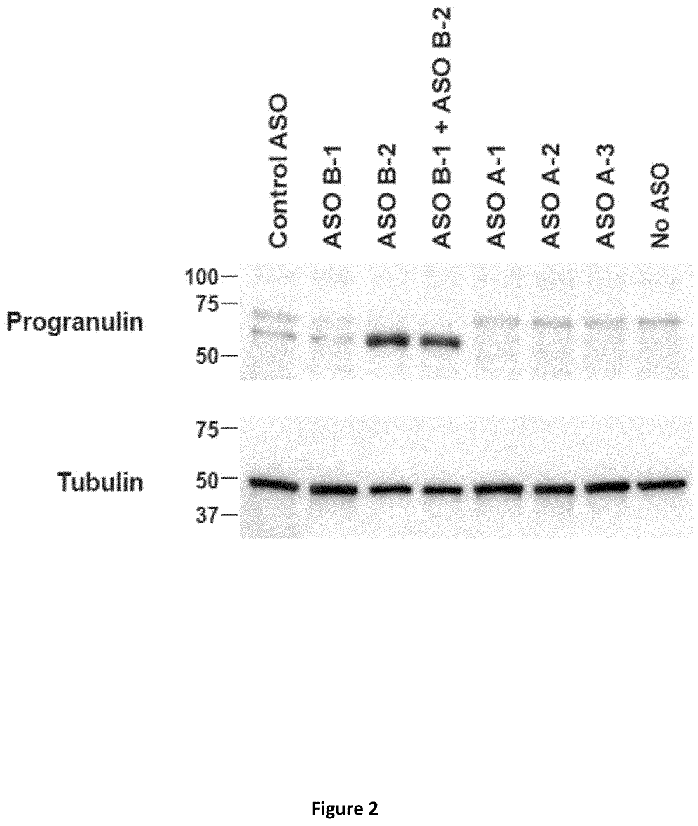

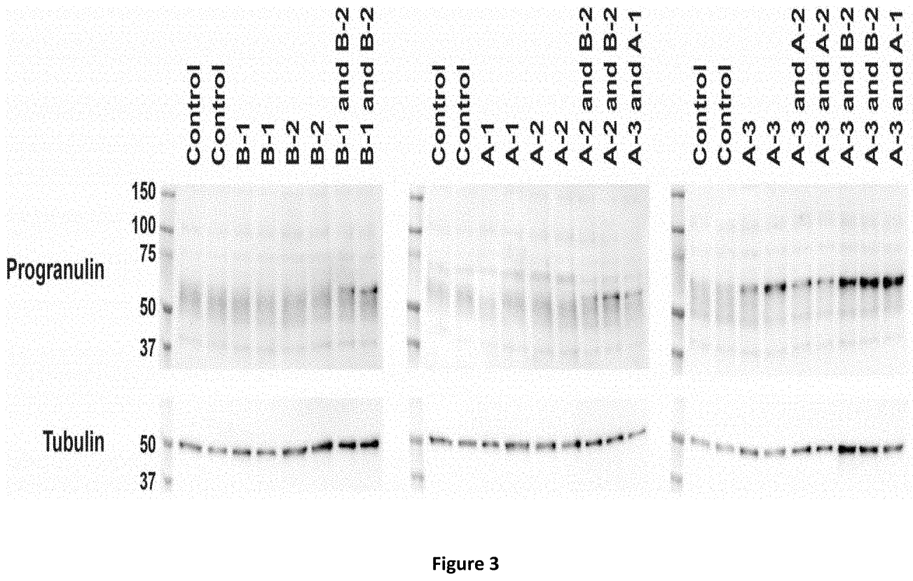

A number of ASOs were tested in cultured cells. The Inventor delivered 100 nM of ASO B-1, ASO B-2, ASO A-1, ASO A-2, and ASO A-3, separately and in combination, to cultures of differentiated SY5Y human neurons by transfection with using Lipofectamine 2000 (see FIGS. 2 and 3). To examine progranulin protein levels, the cells were harvested 2 days post transfection, and samples prepared and applied to western blot analysis. Tubulin was utilized as an internal control to ensure equivalent protein loading between the samples being compared. Controls used were a scrambled sequence ASO that does not target progranulin, or water alone. The Inventor made the surprising discovery that ASO B-2 (FIG. 2) and ASO A-3 (FIG. 3) were found to be particularly effective at increasing progranulin levels in cells, compared to controls. In many cases, administering combinations of ASOs further increased progranulin protein levels compared to administering a single ASO (see FIG. 2, ASOs: B1+B2, A2+B2, A3+A1, A3+A2, A3+B2, and A3+A1).

### Example 2

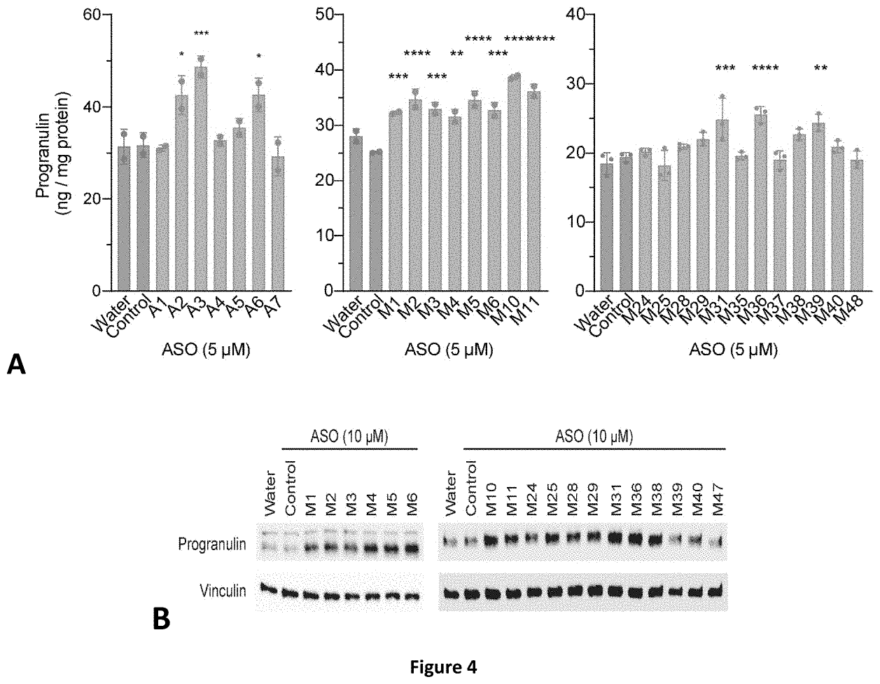

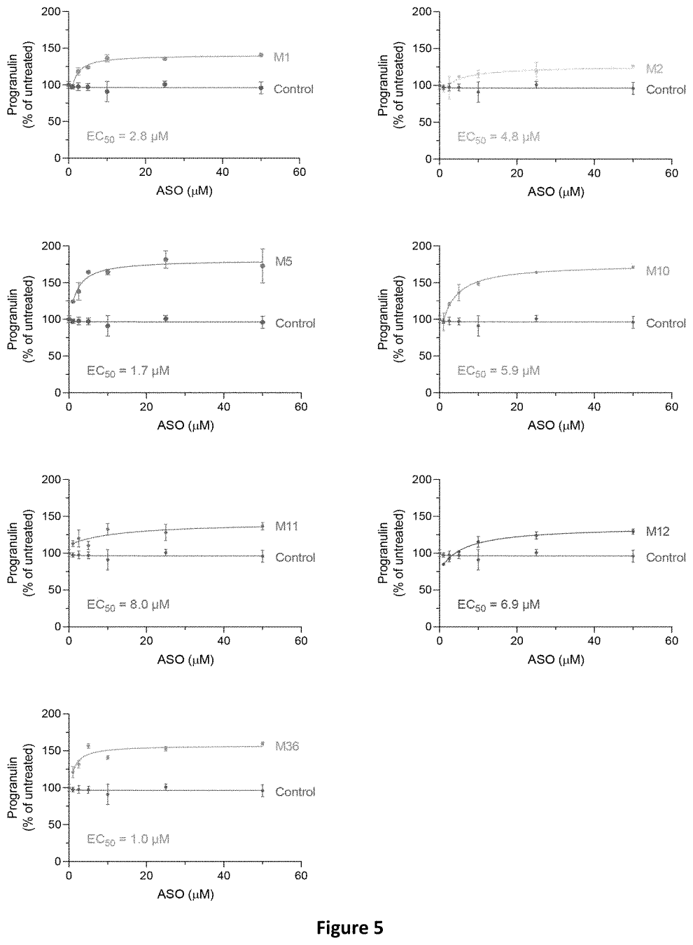

The inventors further demonstrated that their A series and M series ASOs, targeted to the miR-659 and miR-29b binding sites of the GRN mRNA, respectively, possessed the ability to increase progranulin protein levels in a human neural cell line. Human H4 neuroglioma cells were treated with 5 μM of A series or M series ASOs for 24 h and compared to cells treated with either water or a scrambled control ASO. When progranulin protein levels were measured by ELISA, it was observed that out of the 55 ASOs tested, 14 resulted in a statistically significant increase in progranulin protein levels (see FIG. 4A). Western blots confirmed that these positive results were specifically due to increases of human progranulin (10 μM, 24 h) (FIG. 4B). Dose response curves of select ASOs were constructed from the ELISA results after a 24-hour treatment period and EC₅₀ values determined (FIG. 5)

These results demonstrate that A series and M series ASOs result in a specific increase of progranulin protein levels in human neural cells. Construction of dose curves and determination of EC₅₀ allows a practician to anticipate the behavior of these ASOs compared to other known therapeutic agents. The Inventors concluded that select ASOs are effective in increasing human progranulin protein levels in human brain cells.

### Example 3

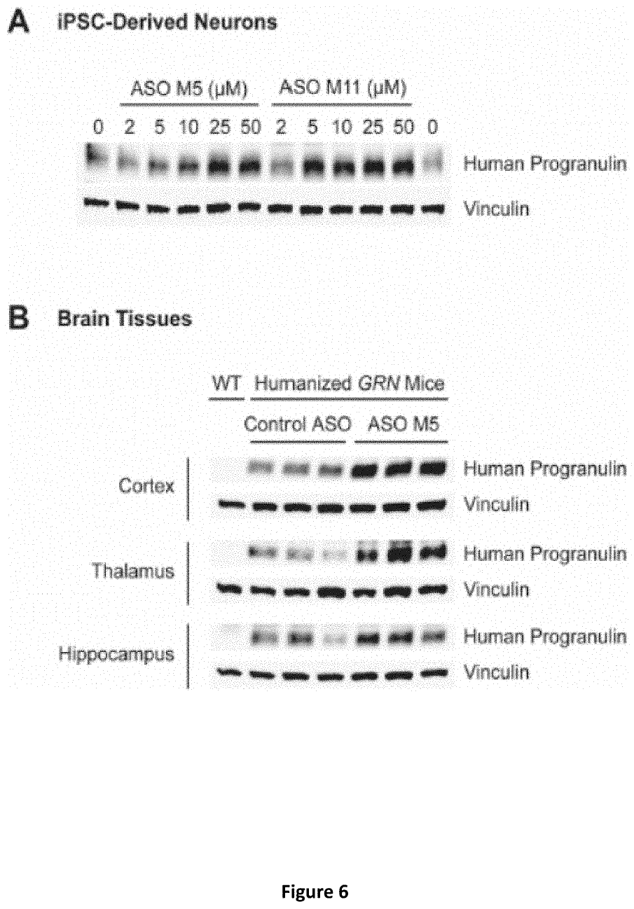

The Inventors further demonstrated that ASOs targeting the miR-29b binding site of GRN mRNA will increased progranulin protein levels in normal human cells both in vitro and in vivo. They demonstrated increased progranulin protein levels in normal human cells in culture and also in the brains of mice which express human progranulin, when administered to these subjects centrally (see FIG. 6).

An increase in progranulin protein levels was observed in a dose-dependent manner when 2 μM to 50 μM of ASOs M5 and M11, was added to the culture media of iPSC-derived cortical neurons for 3 days (see FIG. 6A). The results indicate that the progranulin synthesis may be increased in non-cancerous human neurons using the ASOs of the invention. iPSC cells were used as they are most closely resemble neurons in vivo and are considered the gold standard by researchers in this field.

The Inventors also demonstrated that ASOs administered centrally to a mammal, will increase human progranulin protein levels in the brain. A bolus of 500 μg of ASO M5 per mouse was administered via intracerebroventricular injection (ICV) to transgenic mice engineered with the ability to express human progranulin. After two weeks, brain tissues were analyzed using Western blot techniques. Human progranulin protein levels were increased in the cortex, thalamus and hippocampus of the ASO treated mice compared to mice treated with a scrambled control ASO (see FIG. 6B)

### Prophetic Example

The efficacy of the ASOs will be demonstrated in vivo using the humanized GRN mouse model (PMID: 33636385), in which the mouse Gm gene is deleted. Hemizygous mice carrying one copy of the human GRN gene are genetically similar to patients with heterozygous GRN mutations. For this demonstration, the Inventor will administer an ASO described above, complementary to human progranulin mRNA, which partially overlaps the miR-29b-3p binding site. The ASO will be resuspended in a saline solution and filter sterilized. The Inventor will administer the ASO at a dose of 500 μg per mouse or approximately 12.5 mg/kg, via intracerebroventricular injection, into anesthetized 10-month-old hemizygous GRN^{+/-} mice, as well as homozygous GRN^{+/+} littermate mice, to serve as controls. A scrambled ASO that does not target the progranulin mRNA will be used as a control.

After 2 months, the Inventor will assess cognitive function and behavior by the 3-chamber test of sociability, the tube test of social dominance, the elevated plus maze, cued fear conditioning, and grooming behavior. Subsequently, neuropathology will be assessed by staining the brains for disease-associated markers including Iba1 (for microglia), phosphorylated TDP-43, LAMP1 (for lysosomes), and lipofuscin (autofluorescence), and will also measure progranulin protein levels by western blot analysis. In addition, the Inventor will measure levels of a neuronal death biomarker (neurofilament light chain) in the plasma before and after the procedure. The Inventor will demonstrate that the ASO treatment reverses or halts disease progression in GRN^{+/-} mice, which will be reflected in improvements in cognitive, neuropathological, and biomarker-based measurements (toward the levels observed in the GRN^{+/+} control group). Specifically, the Inventor will demonstrate that the administration of the progranulin-targeting ASOs in GRN^{+/-} mice will lead to increased sociability (which would indicate an improvement in social withdrawal, a common symptom of frontotemporal dementia), increased social dominance, spending less time in the open arms of the elevated plus maze (which would indicate decreased anxiety and/or disinhibition-like behavior, another common symptom of frontotemporal dementia), increased immobile time in the cued fear conditioning test (which would indicate less emotional impairment), and decreased time spent grooming (which would indicate less repetitive and/or compulsive behaviors). In terms of neuropathology, the Inventor will demonstrate that administration of the progranulin-targeting ASOs in GRN^{+/-} mice will result in decreased Iba1 staining (indicated decreased neuroinflammation), decreased phosphorylated TDP-43 staining (which is a pathological feature of progranulin-deficient frontotemporal dementia), decreased LAMP1 staining (indicated decreased lysosomal content, which is increased with progranulin deficiency), and decreased lipofuscin signal (which is increased with progranulin deficiency and generalized lysosomal impairment). Finally, the Inventor will demonstrate that administration of the progranulin-targeting ASOs in GRN^{+/-} mice will result in decreased plasma levels of neurofilament light chain (which would indicate less neuronal death).

All publications and patents cited in this specification are hereby incorporated by reference in their entirety. The discussion of the references herein is intended merely to summarize the assertions made by the authors and no admission is made that any reference constitutes prior art. Applicants reserve the right to challenge the accuracy and pertinence of the cited references.

What is claimed:

1) A composition for increasing progranulin in cells, comprising an antisense oligonucleotide (ASO) complementary to a nucleic acid sequence of the progranulin mRNA, and which binds to, a miR-29b-3p binding site set forth in SEQ ID NO:2 within the progranulin mRNA, and increases progranulin expression when delivered to a cell.

2) The composition of claim 1, with the further modifications of 2′-O-methoxyethyl nucleotides and phosphorothioate linkages.

3) The composition of claim 1, wherein the ASO consists of SEQ ID NO:7.

4) The composition of claim 1, wherein the ASO consists of SEQ ID NO:10.

5) The composition of claim 1, wherein the ASO consists of SEQ ID NO:15.

6) The composition of claim 1, suspended in an injectable solution.

7) The composition of claim 1, wherein the antisense oligonucleotide is at least 14 nucleotides in length.

8) A composition for increasing progranulin in cells, comprising an antisense oligonucleotide, complementary to a nucleic acid sequence of the progranulin mRNA, and which binds to, a miR-659-3p binding site set forth in SEQ ID NO:20, within the progranulin mRNA, and increases progranulin expression when delivered into a cell.

9) The composition of claim 8, with the further modifications of 2′-O-methoxyethyl nucleotides and phosphorothioate linkages.

10) The composition of claim 8, wherein the ASO consists SEQ ID NO:22.

11) The composition of claim 8, wherein the ASO consists of SEQ ID NO:23.

12) The composition of claim 8, suspended in an injectable solution.

13) The composition of claim 8, wherein the antisense oligonucleotide is at least 14 nucleotides in length.

14) A method of treating a subject diagnosed with frontotemporal dementia, the method comprising administering an effective amount of the composition of claim 1.

15) The method of claim 14, wherein administering is via bolus intrathecal injection.

16) The method of claim 14, wherein administering is by intracerebroventricular injection.

17) The method of claim 14, wherein an effective amount is 12.5 mg/kg.

18) The method of claim 14, wherein an effective amount is 1 mg/kg.

19) A method of treating a subject at risk of frontotemporal dementia, the method comprising:
a. determining if the subject is suffering from, or at risk of, frontotemporal dementia by comprehensive testing:
b. the comprehensive testing selected from the group consisting of: genetic testing, neuropsychological examination directed to language, behavior, memory, executive and visual-spatial functions, Mini-Mental State Examination, neuroimaging-based biomarkers, and/or fluid biomarkers in the blood and cerebrospinal fluid including neurofilament light chain and progranulin.
c. if the subject is determined to be suffering from, or at risk of, frontotemporal dementia, administering an effective amount of the composition of claim 1, determined to increase progranulin protein levels by intrathecal or intracerebral injection.


SEQUENCE LISTING (24 sequences)

SEQ ID NO: 1 - DNA, 2130 bp, Homo sapiens
gctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcttaa

SEQ ID NO: 2 - RNA, 21 nt, Homo sapiens
ggacccuguggccaggugcuu

SEQ ID NO: 3 - DNA, 18 bp, Homo sapiens
aaacggggaggggatggc

SEQ ID NO: 4 - DNA, 18 bp, Homo sapiens
gaaacggggaggggatgg

SEQ ID NO: 5 - DNA, 18 bp, Homo sapiens
tgaaacggggaggggatg

SEQ ID NO: 6 - DNA, 18 bp, Homo sapiens
ctgaaacggggaggggat

SEQ ID NO: 7 - DNA, 18 bp, Homo sapiens
actgaaacggggagggga

SEQ ID NO: 8 - DNA, 18 bp, Homo sapiens
cactgaaacggggagggg

SEQ ID NO: 9 - DNA, 18 bp, Homo sapiens
ggtccactgaaacgggga

SEQ ID NO: 10 - DNA, 18 bp, Homo sapiens
gggtccactgaaacgggg

SEQ ID NO: 11 - DNA, 18 bp, Homo sapiens
agcacctggccacagggt

SEQ ID NO: 12 - DNA, 18 bp, Homo sapiens
aaaagcacctggccacag

SEQ ID NO: 13 - DNA, 18 bp, Homo sapiens
gaaaagcacctggccaca

SEQ ID NO: 14 - DNA, 18 bp, Homo sapiens
gggaaaagcacctggcca

SEQ ID NO: 15 - DNA, 18 bp, Homo sapiens
ggatagggaaaagcacct

SEQ ID NO: 16 - DNA, 18 bp, Homo sapiens
gtggatagggaaaagcac

SEQ ID NO: 17 - DNA, 18 bp, Homo sapiens
tgtggatagggaaaagca

SEQ ID NO: 18 - DNA, 18 bp, Homo sapiens
ctgtggatagggaaaagc

SEQ ID NO: 19 - RNA, 16 nt, Homo sapiens
aggccucccuaggccu

SEQ ID NO: 20 - RNA, 20 nt, Homo sapiens
agcaccucccccuaaccaaa

SEQ ID NO: 21 - DNA, 18 bp, Homo sapiens
gtgctagggaggcctgag

SEQ ID NO: 22 - DNA, 18 bp, Homo sapiens
ggtccagggagaatttgg

SEQ ID NO: 23 - DNA, 18 bp, Homo sapiens
gtccagggagaatttggt

SEQ ID NO: 24 - DNA, 18 bp, Homo sapiens
tgctagggaggcctgagc